(19) United States
(12) Patent Application Publication
(10) Pub. No.: US 2004/0152880 A1
(43) Pub. Date: Aug. 5, 2004

(54) Compositions and methods for the reversible capture of biomolecules

(75) Inventor: Jonathan Samuel Minden, Pittsburgh, PA (US)

Correspondence Address: MORGAN, LEWIS & BOCKIUS LLP, 1701 MARKET STREET, PHILADELPHIA, PA, 19103-2921, US

(73) Assignee: Carnegie Mellon University

(21) Appl. No.: 10/719,735

(22) Filed: Nov. 21, 2003

Related U.S. Application Data
(60) Provisional application No. 60/428,560, filed on Nov. 22, 2002.

(51) Int. Cl. C12M 1/34; C07K 14/47

(52) U.S. Cl. 530/410; 435/287.2

(57) ABSTRACT

Substrates (e.g., polymer), and/or solid supports (e.g., glass) having one or more biomolecule-binding compounds covalently bound to the surface of the substrate or solid support reversible covalent attachment of biomolecules thereto.

### CLAIM OF PRIORITY

The present invention claims priority to U.S. Provisional Application Ser. No. 60/428,560, filed Nov. 22, 2002.

### BACKGROUND OF THE INVENTION

Immobilization and separation of biomolecules (e.g., DNA, RNA, peptides, and proteins, to name but a few) through chemical attachment on a solid support or within a matrix material (e.g., hydrogel, e.g., present on a solid support) has become a very important aspect of molecular biology research (e.g., including, but not limited to, DNA synthesis, DNA sequencing by hybridization, analysis of gene expression, and drug discovery).

However, one of the main problems associated with preparing proteins for analysis is the presence of interfering compounds, including but not limited to salts, nucleic acids and lipids. Accordingly, certain techniques have been developed to separate proteins from the interfering compounds.

The reversible blocking of amino groups using maleic anhydride and 2,3-dimethyl maleic anhydride was discussed in a paper by Dixon et al., Biochemical Journal, 109: 312-314 (1968). Similar reactions were also discussed in the paper by Atassi et al., Methods in Enzymology, 49: 546-553 (1972).

Two patents from Kinsella et al. (U.S. Pat. Nos. 4,168,262 and 4,348,479) and two technical reports from the same group (Shetty et al., Biochemical Journal, 191:269-272 (1980); Shetty et al., Journal of Agricultural and Food Chemistry, 30:1166-1172 (1982)) teach a process of separating microbial proteins in bulk from nucleoprotein complexes. The process comprises disruption of the biomass by physical means in the absence of detergents and denaturing reagents. This is followed by centrifugation to remove cell debris, derivatization of the water-soluble proteinaceous material-nucleic acid mixture with an organic dicarboxylic acid anhydride such as citraconic or maleic anhydride, and subjecting the derivatized proteins (freed of insoluble cell debris by centrifugation) to isoelectric precipitation at pH 4.0-4.5. Next, the blocking N-acyl groups are removed by hydrolysis at acid pH, the protein solution is dialyzed to remove salts, and the nucleic acid-depleted bulk proteins are isolated by lyophilization or isoelectric precipitation. It is important to note that the goal of the two Kinsella et al. patents and the Shetty et al. technical reports is to isolate bulk microbial proteins in a form suitable for human consumption from precipitation reactions. The purpose of the N-acylation step is to separate the desired bulk proteins from microbial nucleic acid contaminants.

A device useful for reversibly attaching proteins to a support or other surface is the Reacti-Bind® maleic anhydride plate, commercially available from Pierce Biotechnology Inc., located in Rockford, Ill. The maleic anhydride is bound to a substrate which then can be used to reversibly bind to proteins by altering the environmental pH. The bind and release kinetics of the Reacti-Bind® plates typically takes on the order of hours.

Thus, there is a need for compositions suitable for rapid and efficient protein isolation.

### SUMMARY OF THE INVENTION

In one embodiment, the present invention includes a biomolecule capture device including a substrate having a surface and a maleic anhydride biomolecule-binding compound covalently bound to the surface of the substrate. In one embodiment, the maleic anhydride biomolecule-binding compound has a half life of binding of desired biomolecules of less than 1 hour; and a half life of release of desired biomolecules of less than 1 hour. In one embodiment, the maleic anhydride biomolecule-binding compound includes a dialkyl maleic anhydride. In one embodiment, the biomolecule includes an amine-containing compound. In one embodiment, the biomolecule includes a protein.

In another embodiment, the present invention includes a method of removing and recovering desired biomolecules from a solution via a biomolecule capture device. The method includes the steps of contacting, under basic conditions, a solution containing one or more desired biomolecules with a biomolecule capture device. The biomolecule capture device includes a substrate having a surface and one or more maleic anhydride biomolecule-binding compounds covalently bound to the surface of the substrate. Next, the method includes the step of forming one or more reversible covalent bonds between the biomolecules and the biomolecule-binding compounds, wherein the half life of binding between the biomolecule-binding compounds and the desired biomolecules is less than 1 hour. Next, the biomolecule capture device and biomolecules attached thereto can be washed to remove unwanted biomolecules.

Next, the biomolecule capture device and biomolecules attached or coupled thereto can be exposed to acidic conditions, thereby reversing the covalent bond between the biomolecules and biomolecule-binding compounds and releasing the biomolecules from the biomolecule capture device. Typically in such embodiments, the half-life of release between the biomolecule-binding compounds and the desired biomolecules is less than 1 hour. After the biomolecules have been released, the biomolecules can be recovered and/or isolated.

### BRIEF DESCRIPTION OF THE FIGURES

The present invention will be better understood by examining the following figures which illustrate certain properties of the instant invention wherein:

FIG. 1 shows the formation of a dialkyl maleic anhydride derivative and the coupling of that derivative to a support for use in the present invention;

FIG. 2 shows a reversible binding reaction between a composition of the present invention and a biomolecule (depicted as a protein) and shows a general linkage of a dialkyl maleic anhydride to a substrate, wherein the linkage can include, but is not limited to, an amide bond.

### DETAILED DESCRIPTION OF THE INVENTION

In one embodiment, the present invention is directed to isolation of a biomolecule. Preferably, the biomolecule includes an amine. In one embodiment, the amine-containing compound is a protein.

Typically, isolation of a biomolecule such as an amine-containing compound (e.g., proteins) is accomplished by time consuming protein precipitation and washing techniques. After washing, the biomolecule can then be redissolved for analysis.

Unfortunately, not all biomolecules precipitate completely, and not all biomolecules redissolve completely. Further, the washing techniques can fail to remove significant amounts of undesirable substances such as unwanted nucleic acids, salts, lipids and other cell debris. Therefore, significant quantities of desired biomolecules may be lost or difficult to analyze.

Furthermore, other isolation and separation techniques involve non-covalent interactions with biomolecules (e.g., proteins), as most biomolecules (e.g., proteins) have a very wide range of chemical characteristics that can be utilized for non-covalent binding events. However, none of these non-covalent methods can capture all or substantially all biomolecule (e.g., protein) species in a cell or solution.

The present invention includes compositions and methods suitable for the rapid recovery and isolation of biomolecules such as amine containing compounds (e.g., proteins) from a solution. The present invention captures biomolecules (e.g., proteins) on a reversible matrix. By forming a reversible covalent bond between the matrix and the biomolecules, a high percentage of the biomolecules can be retained on the matrix after extensive washing to remove contaminants. The linkage between the matrix and biomolecules can then be reversed to release the captured proteins which can be isolated, thereby improving analysis accuracy and efficiency of the biomolecule.

A composition of the present invention includes two components: 1) a substrate or surface suitable for contact with a desired biomolecule to be isolated (e.g., proteins) and capable of forming covalent bonds with another compound, and 2) one or more biomolecule-binding compounds attached or bound to a surface of the substrate, wherein the biomolecule-binding compound is capable of forming reversible covalent bonds with a biomolecule. Each of these components will be described further below. In some embodiments that involve amide bonds, it should be noted that typically the strength of the covalent bond between the substrate and biomolecule-binding compound equals the strength of the bond between the biomolecule-binding compound and the biomolecule.

1. Substrates

The present invention includes the use of a substrate, including inorganic crystals, inorganic glasses, inorganic oxides, metals, and/or polymers including but not limited to a hydrogel, and/or polymer hydrogel array, each containing one or more reactive sites for the attachment of a biomolecule-binding compound, described in more detail below. In one embodiment, reactive sites can include, but are not limited to, exposed amino groups, such that covalent amide linkages can be formed between the substrate and the biomolecule-binding compound, as described further below.

1(a). Substrates for Use With the Present Invention

Desirably, a suitable substrate is a polymeric substrate or “polymer” for use in the invention. Suitable polymers can be any polymer or mixture of polymers, including but not limited to, hydrophilic polymers, suitable for use with amino groups and/or for use with proteins. In certain embodiments the substrate can include one or more of the following polymers: polyamide, polyacrylamide, polyester, polycarbonate, hydroxypropylmethylcellulose, polyvinylchloride, polymethylacrylate, polystyrene and copolymers of polystyrene, polyvinyl alcohol, polyacrylic acid, polyethylene oxide and combinations thereof.

The substrate can also be one or more substances selected from a list including, but not limited to, collagen, dextran, cellulose or cellulosics, calcium alginate, latex, polysulfone, agarose, including but not limited to aminohexyl agarose and aminododecyl agarose, and glass.

Any of the above described substrates can be chemically modified using techniques known in the art to provide suitable reactive sites for the attachment of a biomolecule-binding compound, if such suitable sites are not already available by virtue of the substrate.

1(b). Shapes and Application of the Substrate

One or more of the polymers described above may be formed into any regular or irregular shape, provided that one or more reactive sites for the attachment of a biomolecule-binding compound remains exposed.

1(b)1. Optional Solid Supports

In one embodiment, the polymers described above can also be optionally applied to a “solid support.” The “solid support” according to the invention can be any type of solid support. In one embodiment, the solid support can be hydrophilic.

If a solid support is included as part of the invention, in some embodiments the solid support is a material selected from the group including, but not limited to, nylon, polystyrene, glass, latex, plastics, polypropylene, and activated cellulose. Other materials include films, silicon, modified silicon, ceramic, plastic, other appropriate polymers such as (poly)tetrafluoroethylene, or (poly)vinylidenedifluoride.

The solid support can be any shape or size, and can exist as a separate entity or as an integral part of any apparatus (e.g., bead, cuvette, plate, vessel, and the like). It further is assumed that appropriate treatment of the solid support (e.g., glass) will be undertaken to provide adherence of one or more of the polymers described above to the surface of the solid support, e.g., with gamma-methacryl-oxypro 1-trimethoxysilane (“Bind Silane”, Pharmacia), or other appropriate means in cases where the polymer is present on a solid support. In one embodiment, covalent linkage of a polyacrylamide hydrogel to the solid support can be done as described in European Patent Application 0 226 470 (incorporated by reference).

In one embodiment, the biomolecule-binding compound is linked to a substrate, which is applied to a solid support, either before, after, or during linkage of the biomolecule-binding compound to the substrate. In another embodiment, the biomolecule-binding compound can be linked directly to a solid support, forgoing the use of a substrate.

1(b)2. Shape

Desirably the polymer and/or solid support (if present) is a material (i.e., is present in a form) selected from the group consisting of a bead or microsphere, mesh, membrane, microwell, centrifuge tube, and plate or slide.

Commercial examples of microspheres, which are described as including a purified collagen, include ICN Collagen Beads and Cellex Biosciences macroporous microspheres. Suitable microspheres can have a porous or smooth consistency, and typically have an approximately spherical shape with a diameter of approximately 0.1 to 2 mm. Of course, the shape and size of microspheres from any particular lot or preparation will vary within manufacturing tolerances. Suitable agarose beads can be readily obtained from Sigma-Aldrich Chemical Corp. St. Louis, Mo.

2. Biomolecule-Binding Compound

The biomolecule-binding compound can be any compound that forms a reversible covalent bond with a biomolecule. As used herein, “biomolecule” includes, but is not limited to, any amine containing compound such as amino acids and proteins as well as nucleic acids and lipids. In certain embodiments, the biomolecule-binding compound can be coupled to a support, typically by a covalent bond, as described above, and yet retain the ability to form a reversible covalent bond with a biomolecule. In certain embodiments, the biomolecule-binding compound includes a maleic anhydride compound having the desired biomolecule binding and release characteristics, as described hereinbelow. In one embodiment, the maleic anhydride includes a dialkyl maleic anhydride.

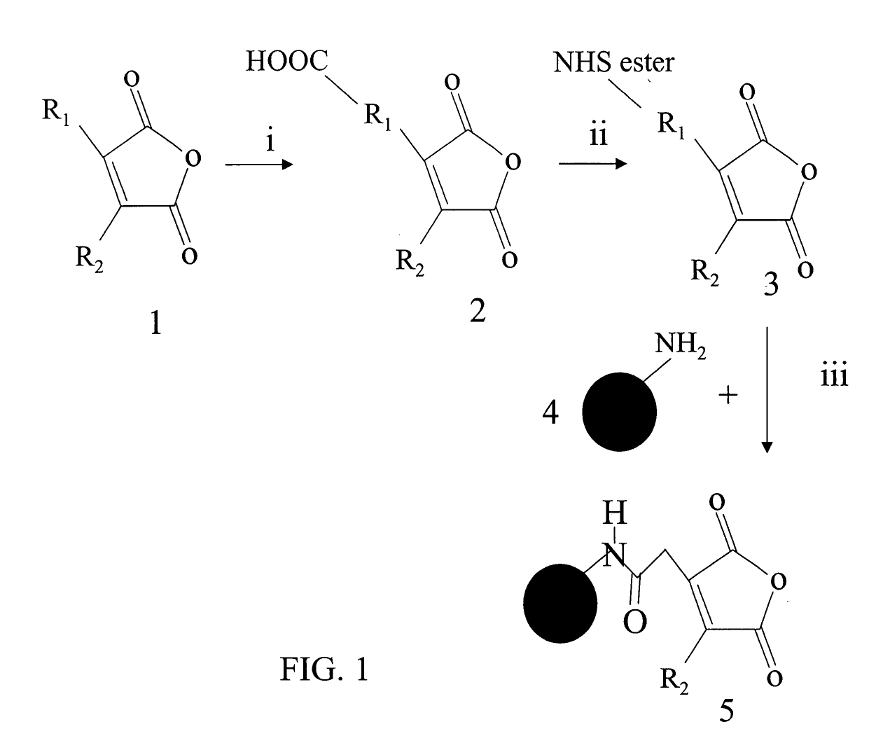

Typically, one or more alkyl groups can be coupled to the maleic anhydride at the molecular “2” and “3” positions, thus forming a dialkyl maleic anhydride, as shown in FIG. 1. Accordingly, in one embodiment of the present invention the biomolecule-binding compound includes a dialkyl maleic anhydride.

In one embodiment, the present invention includes a maleic anhydride having a first alkyl group at the molecular “2” position, and a second alkyl group at the “3” position. The alkyl group at the “2” position, designated as R2, can be any alkyl group, including but not limited to alkanes, including methyl, ethyl, propyl, butyl and pentyl groups as well as unsaturated alkyl groups including alkenes and alkynes. Other alkyl groups are well known in the art, including benzyl functional groups. The functional group can also be a hydroxyl group, as well as those functional groups set forth in Organic Chemistry, 3rd Ed., John McMurray, Brooks/Cole Publishing Co. (1992), the entire content of which is hereby incorporated by reference.

The other alkyl group at the molecular “3” position, designated as R1, can also be any alkyl group as described with respect to the R2 group above, and is can be selected from methyl, ethyl, propyl, butyl and pentyl compounds, but can also be any alkyl group, including but not limited to alkanes, including methyl, ethyl, propyl, butyl and pentyl groups as well as unsaturated alkyl groups including alkenes and alkynes. However, R1 should be a group that is capable of forming a covalent bond with an exposed reactive site of the substrate and/or support, either before or after any modifications to the R1 group, as described below.

In yet another embodiment, the dialkyl maleic anhydride compound includes 2,3 dimethyl maleic anhydride; 2-methyl, 3-ethyl maleic anhydride; 2,3 diethyl maleic anhydride and derivatives thereof. In one embodiment, the dialkyl maleic anhydride is 2,3 dimethyl maleic anhydride. Maleic anhydride and dimethyl maleic anhydride can be obtained from Sigma-Aldrich Chemical Co., St. Louis, Mo.

3. Method of Making a Composition of the Present Invention

To couple a maleic anhydride to a support, one or more of the above described maleic anhydrides can be chemically modified to form a derivative of a maleic anhydride. Such derivatives include those derivatives suitable for forming covalent bonds between the derivative and a substrate and/or support, as described further below. In one embodiment, a maleic anhydride is coupled to a support and/or substrate by way of exposed reaction sites on the substrate and/or support, thus forming a device of the present invention.

In one embodiment, a dialkyl maleic anhydride, including but not limited to a dialkyl maleic anhydride described hereinabove, can be chemically modified to form a 2 alkyl 3 carboxyalkyl maleic anhydride derivative, as shown in FIG. 1. The carboxyalkyl (or carboxyl) group can then be chemically modified into an N-hydroxysuccinimidyl (NHS) ester as also shown in FIG. 1. The NHS ester can then be contacted with any suitable substrate and/or support having exposed reactive sites, e.g., exposed amino groups, to form linkages between the dialkyl maleic anhydride and the substrate. The reversible binding properties of the biomolecule-binding compound are at most minimally affected by linkage to the substrate. In some preferred embodiments, care should be taken to prevent the removal of the double bond that exists between the “2” and “3” carbon atoms on a dialkyl maleic anhydride.

In another embodiment, a dimethyl maleic anhydride can be used as a biomolecule-binding compound. The methyl group at the “3” position can be chemically modified to form a carboxyalkyl group by the following steps, shown with reference to FIG. 1. In step (i), N-bromosuccinimide (NBS), benzoyl peroxide, CCl₄, can be contacted with the dimethyl maleic anhydride (1) for about 10 hours. Then, dimethyl malonate, NaH, and C₆H₆, can be contacted with the compound for about 8 hours. Next, HCl can be added and any reactions can be permitted to proceed for about 12 hours, thereby forming 2-methyl, 3-carboxymethyl maleic anhydride (2).

The carboxymethyl group can then be chemically modified into an N-hydroxysuccinimidyl (NHS) ester (3). In step (ii), O-(N-Succinimidyl)- N,N,N′,N′-tetramethyluronium tetrafluoroborate (TSTU), and DMF, can be contacted with the 2-methyl, 3-carboxymethyl maleic anhydride (2) for about 30 minutes, thus forming an NHS ester (3).

In step (iii) the NHS ester (3) can be contacted with the exposed reactive site of a substrate (4) at a pH of about 7 for about 12 hours, thereby forming an amide linkage between the anhydride and the substrate (4) and thus forming a device (5) of the present invention.

Because the reactive nature of maleic anyhydrides, the covalent attachment of the biomolecule-binding compound to the support should occur under conditions that favor the formation of a bond between the chemically modified portion of the maleic anhydride and the exposed reactive site of the support and not favor bond formation between the anhydride and the exposed reactive site. In one embodiment, the covalent coupling of a NHS ester to an exposed amine reactive site occurs at a pH of about 7.

The above described process can be further optimized as necessary or desired in terms of reaction conditions, duration of contact, length of carboxyl groups, alkyl groups, amount of reactive sites available on the substrate, etc.

4. Method of Use

A substrate having a biomolecule-binding compound covalently linked thereto can be used to separate one or more desired biomolecules from a solution. In one embodiment, the biomolecule includes any amine-containing compound. In one embodiment, the biomolecule contains at least one lysine. Other suitable biomolecules can include amino acids, proteins, nucleic acids or lipids. In a preferred embodiment, the biomolecule includes a protein. As described further herein, the biomolecule is primarily depicted as a protein, however the scope of the invention should not be limited thereto.

In an embodiment of the present invention, a dialkyl maleic anhydride bound to a support can be effective to capture, remove and/or recover protein from a solution or other suitable medium that includes desired biomolecule (e.g., proteins) or other materials.

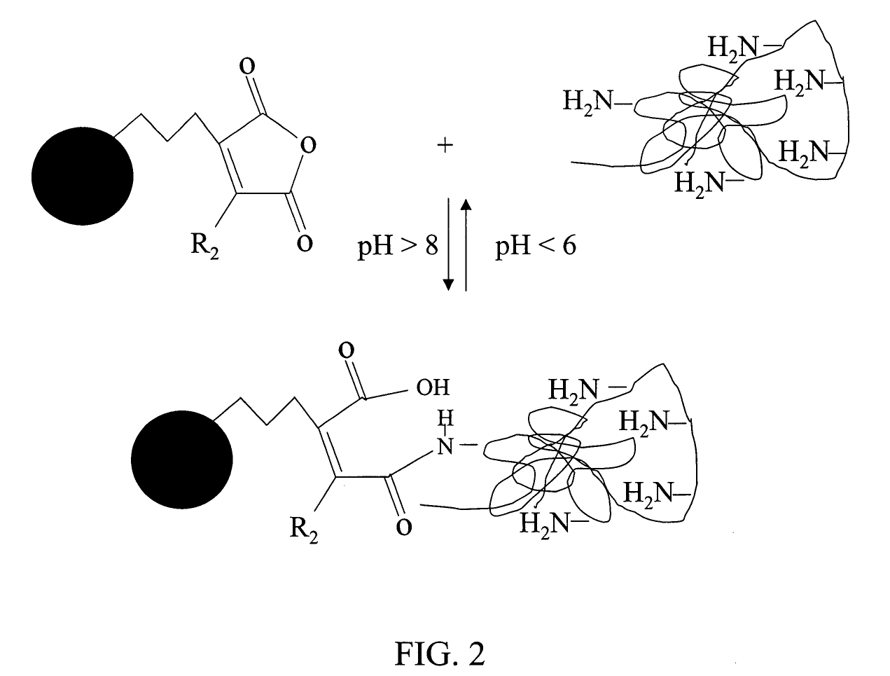

Specifically, a dialkyl maleic anhydride bound to a substrate and/or support can be exposed or contacted with a solution containing one or more biomolecules (e.g., proteins). The lysine amino acids in the protein form reversible covalent bonds with the dialkyl maleic anhydride at a first environmental pH, typically a basic pH, and in certain embodiments the pH can be about 8.0, as shown in FIG. 2, thereby binding to and capturing the proteins. In one embodiment, lysine reacts with the cyclic anhydride to form an amide linkage between the amine and carbonyl group. This opens the ring, releasing a carboxylate on the other end of the opened ring, as shown in FIG. 2. Once bound, the support and the proteins bound thereto can be vigorously washed and cleaned to remove any unwanted biomolecules, e.g., nucleic acids and/or lipids and/or salts. Once cleaned, using such techniques understood in the art in light of the teachings herein, the covalent bonds between the biomolecule-binding compound and proteins can be reversed by adjusting the environmental pH to a second pH which is different from the first pH, thereby releasing the bound proteins. Typically the second pH is lower than the first pH, more typically the second pH is an acidic pH, and in certain embodiments the second pH can be about 6.0, as shown in FIG. 2.

It should be noted that the capture and release pH depends upon the particular biomolecule-binding compound used, and in some instances the binding environmental pH can be lower than the release environmental pH. The determination of such binding and release pHs is within the ability of one of skill in the art, typically about 3 to 11.

The proteins can then be removed and/or recovered from the substrate using techniques well known in the art, e.g., elution. Binding and removal can be performed at any temperature, however a temperature in the range of about 10 to 35 degrees Celsius, typically about 25 degrees Celsius can be used. In another embodiment of the present invention, binding and removal occurs at room temperature.

In one embodiment, during the capture and/or retention phase (e.g., at a first environmental pH) the biomolecule-binding compound bound to the substrate of the present invention can capture and/or retain at least 10% to 99% of the total protein in a solution. In another embodiment at least 10%, 25%, 50%, 60%, 70%, 80% or 90% of the total protein in a solution can be captured and/or retained. In another embodiment, the present invention can capture and/or retain at least 95% to 99% of the total protein in a solution. The binding of a biomolecule to a biomolecule-binding compound typically depends upon the total number of available binding sites. In one embodiment of the present invention, typically about 2% to 10%, more typically 2% to 5% of the total lysines available for binding can be bound, or about 1 lysine per biomolecule (e.g., protein).

In another embodiment, during the release and/or recovery phase (e.g., when the environmental pH is changed to a different pH than that used during the capture and/or retention phase), the biomolecule-binding compound of the present invention can release and/or permit recovery of at least 10% to 99% of the total biomolecules (e.g., proteins) in the original solution. In another embodiment, at least 10%, 25%, 50%, 60%, 70%, 80% or 90% of the total biomolecules (e.g., proteins) in the original solution can be released and/or recovered. In another embodiment, the present invention can release and/or permit recovery of at least 95% to 99% of the total biomolecules (e.g., proteins) in the original solution.

Accordingly, the present invention provides for the efficient capture, release and/or recovery of biomolecules from a solution. Further, because of the nature of the reversible covalent bond of the present invention, the amount of time required for the covalent bonds to form and/or reverse can be reduced from a half life of hours (e.g. about 2 hours when a monoalkyl maleic anhydride is used as a biomolecule-binding compound) to minutes (e.g., about 2 to 30 minutes).

Specifically, in certain embodiments of the present invention, the amount of time required to covalently bind half of the proteins in a solution is defined herein as the “binding half life.” The present invention can reduce the binding half life from hours to minutes. Similarly, in certain embodiments of the present invention, the amount of time required to release half of the protein which is covalently bound to the biomolecule-binding compound is defined herein as the release half life. The present invention can reduce the release half life from hours to minutes.

In one embodiment, the biomolecule-binding compound bound to the substrate has a biomolecule-binding half life of less than 1 hour. In one embodiment, the biomolecule-binding compound bound to the substrate has a biomolecule-binding half life of less than 45 minutes. In one embodiment, the biomolecule-binding compound bound to the substrate has a biomolecule-binding half life of less than about 30 minutes. In one embodiment, the biomolecule-binding compound bound to the substrate has a biomolecule-binding half life of less than about 20 minutes. In yet another embodiment, the biomolecule-binding compound bound to the substrate has a biomolecule-binding half life of less than about 10 minutes. In yet another embodiment, the biomolecule-binding compound bound to the substrate has a biomolecule-binding half life of less than about 5 minutes. In one embodiment, the biomolecule-binding compound bound to the substrate has a biomolecule-binding half life of less than about 2 minutes. As used herein, the term “about” means plus or minus 10% of the value referenced, thus “about 10” means 9 to 11.

In one embodiment, the biomolecule-binding compound bound to the substrate has a biomolecule release half life of less than about 1 hour. In one embodiment, the biomolecule-binding compound bound to the substrate has a biomolecule release half life of less than about 45 minutes. In one embodiment, the biomolecule-binding compound bound to the substrate has a biomolecule release half life of less than about 30 minutes. In one embodiment, the biomolecule-binding compound bound to the substrate has a biomolecule release half life of less than about 20 minutes. In yet another embodiment, the biomolecule-binding compound bound to the substrate has a biomolecule release half life of less than about 10 minutes. In yet another embodiment, the biomolecule-binding compound bound to the substrate has a biomolecule release half life of less than about 5 minutes. In one embodiment, the biomolecule-binding compound bound to the substrate has a biomolecule release half-life of less than about 2 minutes.

In one embodiment, the present invention can capture about 50% of the proteins in a solution in less than about 10 minutes, about 75% in less than about 20 minutes and about 87.5% of the proteins in a solution in less than about 30 minutes. In another embodiment the present invention can release about 50% of the proteins covalently bound to the substrate in less than about 10 minutes, about 75% in less than about 20 minutes and about 87.5% of the proteins bound to the substrate in less than about 30 minutes.

The present invention can also be used for labeling and subsequent treatment or processing of biomolecules. Specifically, after a biomolecule is bound to the substrate and/or support and before, during or after washing, the biomolecule can be modified, e.g. by labeling, phosphorylation, biotinylation, etc., while the biomolecule is immobilized on the substrate and/or support. The modified biomolecule can then be recovered as described above, e.g., by elution.

It should also be noted that some biomolecules, and in particular certain proteins, may also resist reversal of the covalent bond between the protein and biomolecule-binding compound. This group of proteins is expected to be relatively small.

It will be appreciated by persons skilled in the art that numerous variations and/or modifications may be made to the invention shown in the specific embodiments without departing form the spirit and scope of the invention as broadly described. Further, each and every reference cited above is hereby incorporated by reference as if fully set forth herein.

What is claimed is:

1. A biomolecule capture device comprising:
(a) a substrate having a surface;
(b) a maleic anhydride biomolecule-binding compound covalently bound to the surface of the substrate, the maleic anhydride biomolecule-binding compound having a half life of binding of desired biomolecules of less than 1 hour; and a half life of release of desired biomolecules of less than 1 hour.

2. The biomolecule capture device of claim 1, the substrate comprising a polymer having exposed reactive sites on the surface.

3. The biomolecule capture device of claim 2, the substrate comprising one or more of polyamide, polyacrylamide, polyester, polycarbonate, polyethylene oxide, hydroxypropylmethylcellulose, polyvinylchloride, polymethylacrylate, polystyrene and copolymers of polystyrene, polyvinyl alcohol, polyacrylic acid, collagen, dextran, cellulose, calcium alginate, latex, polysulfone, agarose, aminohexyl agarose, aminododecyl agarose, and glass.

4. The biomolecule capture device of claim 2, the substrate comprising aminohexyl agarose or aminododecyl agarose.

5. The biomolecule capture device of claim 1, the maleic anhydride biomolecule-binding compound comprising a dialkyl maleic anhydride.

6. The biomolecule capture device of claim 1, the maleic anhydride biomolecule-binding compound comprising dimethyl maleic anhydride, methyl ethyl maleic anhydride, or diethyl maleic anhydride.

7. The biomolecule capture device of claim 1, comprising a solid support.

8. The biomolecule capture device of claim 1, the desired biomolecule comprising an amine containing compound.

9. The biomolecule capture device of claim 8, the amine containing compound comprising a protein.

10. A method of removing and recovering desired biomolecules from a solution comprising the steps of
(a) contacting, under basic conditions, a solution containing one or more desired biomolecules with a biomolecule capture device comprising a substrate having a surface and one or more maleic anhydride biomolecule-binding compounds covalently bound to the surface of the substrate;
(b) forming one or more reversible covalent bonds between the biomolecules and the biomolecule-binding compounds, wherein the half life of binding between the biomolecule-binding compounds and the desired biomolecules is less than 1 hour;
(c) washing the biomolecule capture device and biomolecules attached thereto to remove unwanted biomolecules;
(d) exposing the biomolecule capture device to acidic conditions, thereby reversing the covalent bond between the biomolecules and biomolecule-binding compounds thereby releasing the biomolecules from the biomolecule capture device, wherein the half life of release between the biomolecule-binding compounds and the desired biomolecules is less than 1 hour; and
(e) recovering the desired biomolecules.

11. The method of claim 10, the desired biomolecules comprising proteins.

12. The method of claim 10, the maleic anhydride biomolecule-binding compound comprising a dialkyl maleic anhydride.

13. The method of claim 10, the maleic anhydride biomolecule-binding compound comprising dimethyl maleic anhydride, methyl ethyl maleic anhydride, or diethyl maleic anhydride.

14. The method of claim 10, wherein the half life of binding between the biomolecule-binding compounds and the desired biomolecules is less than 30 minutes.

15. The method of claim 10, wherein the half life of release between the biomolecule-binding compounds and the desired biomolecules is less than 30 minutes.

16. The method of claim 10, the biomolecule capture device having a bead shape and is located in a column.

17. The method of claim 10, the desired biomolecule comprising an amine containing compound.

18. The method of claim 17, the amine containing compound comprising a protein.

19. A method of making a biomolecule capture device comprising:
(a) providing a substrate having one or more exposed reactive sites thereon;
(b) providing a dialkyl maleic anhydride;
(c) converting one alkyl group of the dialkyl maleic anhydride to a carboxyalkyl group;
(d) converting the carboxyalkyl group into a N-hydroxysuccinimidyl ester;
(e) contacting the dialkyl maleic anhydride with the substrate having the exposed reactive sites; and
(f) forming a covalent bond between the substrate and dialkyl maleic anhydride.

20. The method of claim 19, the substrate comprising the form of a bead.

21. The method of claim 19, wherein the substrate is on a solid support.

22. The method of claim 19, the substrate comprising one or more of polyamide, polyacrylamide, polyester, polycarbonate, polyethylene oxide, hydroxypropylmethylcellulose, polyvinylchloride, polymethylacrylate, polystyrene and copolymers of polystyrene, polyvinyl alcohol, polyacrylic acid, collagen, dextran, cellulose, calcium alginate, latex, polysulfone, agarose, aminohexyl agarose, aminododecyl agarose, and glass.

23. The method of claim 19, the dialkyl maleic anhydride comprising dimethyl maleic anhydride, methyl ethyl maleic anhydride, or diethyl maleic anhydride.

24. A method of making a biomolecule capture device comprising:
(a) providing a substrate having one or more exposed amine reactive sites thereon;
(b) providing a dialkyl maleic anhydride having a N-hydroxysuccinimidyl ester at the 3 position and an alkyl group at the 2 position of the maleic anhydride;
(e) contacting the maleic anhydride with the substrate having the exposed amine reactive sites; and
(f) forming one or more covalent amide bonds between the substrate and maleic anhydride having a N-hydroxysuccinimidyl ester at the 3 position and an alkyl group at the 2 position of the maleic anhydride.

25. A biomolecule capture device comprising:
(a) a substrate having a surface;
(b) a dialkyl maleic anhydride biomolecule-binding compound covalently bound to the surface of the substrate.

26. The biomolecule capture device of claim 25, the substrate comprising aminohexyl agarose or aminododecyl agarose.

27. The biomolecule capture device of claim 25, the dialkyl maleic anhydride biomolecule-binding compound comprising dimethyl maleic anhydride, methyl ethyl maleic anhydride, or diethyl maleic anhydride.

28. The biomolecule capture device of claim 25, the substrate comprising a polymer having exposed reactive sites on the surface.

29. The biomolecule capture device of claim 25, the substrate comprising one or more of polyamide, polyacrylamide, polyester, polycarbonate, polyethylene oxide, hydroxypropylmethylcellulose, polyvinylchloride, polymethylacrylate, polystyrene and copolymers of polystyrene, polyvinyl alcohol, polyacrylic acid, collagen, dextran, cellulose, calcium alginate, latex, polysulfone, agarose, aminohexyl agarose, aminododecyl agarose, and glass.
